(19) United States
(12) Patent Application Publication
(10) Pub. No.: US 2018/0021556 A1
(43) Pub. Date: Jan. 25, 2018

(54) PAINLESS DRUG IMPLANTER

(71) Applicant: Chee Yen LIM, Singapore (SG)

(72) Inventors: Chee Yen LIM, Singapore (SG); Yong Wei CHIA, Singapore (SG); Wei Siong LEE, Singapore (SG)

(21) Appl. No.: 15/547,550

(22) PCT Filed: Feb. 13, 2015
(86) PCT No.: PCT/SG2015/050022

§ 371 Date: Jul. 31, 2017

(51) Int. Cl. A61M 37/00; A61M 5/31

(52) U.S. Cl. CPC A61M 37/0069; A61M 37/0015; A61M 5/3134; A61M 5/3148; A61M 5/158

(57) ABSTRACT

The present invention relates to a drug implant device which delivers a drug load to the body painlessly. The present invention achieves the painless drug implantation by adopting two principles: (1) rapid perpendicular insertion of fine cannula is painless and that (2) pain is incurred only when the occupied volume caused by the implant process is increased. Therefore, instead of inserting a cannula and injection a volume of drug, which increases the occupied volume of the injection process due to additional volume of the drug, the present invention retracts the cannula in order to dispose the drug into the body. The retraction of cannula does not increase the occupied volume therefore incurs no pain. In the preferred embodiment, the drug implant device (100) comprises a cannula (300) with a bevelled tip, a drug load (320) and an inner rod (340), wherein the drug load (320) and the inner rod (340) are slidably disposed within the cannula (300), and that the drug load (320) is disposed at the bevelled end of the cannula (300) and that the inner rod (340) is disposed adjacent to the drug load (320).

### FIELD OF THE INVENTION

The present invention relates to intradermal, subcutaneous or intramuscular drug delivery. More particularly, the present invention relates to rapidly implanting an active pharmaceutical ingredient into the body painlessly.

### BACKGROUND OF THE INVENTION

Administration of drug into human bodies is one of the most important interventions in medical treatment and disease prevention. There are two general drug administration routes that are commonly practiced, namely enteral and parenteral drug administration. The enteral administration route involves the esophagus, stomach, and intestines and parenteral administration route involves injection and infusion by needles. The most common enteral administration is oral administration (i.e. eating or drinking the drug). The advantage of this route is that it is easy to do, but the disadvantage is that the drug may be disintegrated or degraded by the gastro-intestinal tract and therefore much higher dose is needed, which may be toxic to the body. For this reason, all biologics are not suitable to be administered orally because the gastro-intestinal tract will digest or degrade them.

On the other hand, parenteral administration generally involves injection or infusion via cannulas, which includes subcutaneous, intradermal, intramuscular and intravenous injections using stainless steel needles. Parenteral injections avoid the gastro-intestinal tract so is ideal for biological drugs such as peptides, hormones, vaccines and antibodies. Its other advantages are fixed and accurate doses and short administration times, normally within 3-5 seconds for injections (except for infusion). Its main disadvantage is the pain incurred during the short period of time. The pain associated with parenteral injections is caused by two separate actions, namely the insertion of needle and the injection of liquid into the body. There is a long-felt need to eliminate the pain caused by parenteral injections.

Microneedles were invented to provide a solution to eliminate the pain due to parenteral injections. There are two kinds of microneedles: the first kind is solid microneedles in which drug is loaded on the microneedles and is delivered to the body when the microneedles penetrate into the skin; another kind is hollow microneedles in which drugs are injected into the skin through the hollow microneedles. The insertion of solid and hollow microneedles is quite painless due to the micron-size of the microneedles. But due to the size of the solid microneedles, the amount of the drug which can be loaded on the microneedles is very limited.

As a matter of fact, there are many problems associated with delivering drug using solid microneedles. For example, there are commonly two ways to load the drugs on the solid microneedles: (a) the drugs may be coated on the surface of non-dissolving microneedles or (b) the drugs may be mixed with dissolving materials such as hyaluronic acid and moulded into dissolvable microneedles. The solid microneedles by the first method, apart from having limited drug loading capacity, is not able to deliver complete dose because the coated drug will come off the needles' surface and stay outside the skin. The U.S. Pat. No. 8,361,022 B2 awarded to Alza Corp. and U.S. Pat. No. 7,846,488 B2 awarded to 3M Innovative Properties Company reported the inventions involving the coating of drugs on solid microneedles.

The second method to load drugs on the solid microneedles is to mix the drug with the dissolving material and mould the mixture into dissolvable microneedles. The dissolving materials may be hyaluronic acid, chitosans, hydrogels and other polymers that dissolve upon making contact with the skin. Among other problems, the paramount issue is that the drug mixture becomes a new form of drug and therefore requires separate clinical approval if the drugs involved are controlled by the regulatory bodies. This imposes great impedance for the technology to reach the market because the clinical trials involved are lengthy and expensive, and each drug has to undergo a new drug approval process. U.S. Pat. No. 8,167,852 B2 awarded to Cosmed Pharmaceutical Co. Ltd and U.S. Pat. No. 8,506,980 B2 awarded to Bioserentach Co. Ltd reported inventions related to this type of microneedles.

We mentioned earlier that drugs can also be injected via hollow microneedles. In this case, the insertion of hollow microneedles into the skin is quite painless and they can continuously inject liquid drugs into the skin, which in this case increase tremendously the deliverable amount of the drug. But the injection of liquid via hollow microneedles into the skin causes pain, and since the hollow microneedles' injection rate is much lower than that of the conventional cannulas, the pain is felt much longer (the delivery time for 0.5 ml is 5-30 minutes for hollow microneedles compared to 3-5 seconds for cannulas). Therefore, microneedles do not totally solve the pain associated with parenteral injections. PCT application WO 2011/014514A1, which was filed by 3M Innovative Properties Company reported such a technology.

There is yet another new technology developed by Glide Pharma involves inserting a drug load at a few metre per second with the help of a pioneer projectile, which is made of a biodegradable material. Typical drug may not have the required hardness for penetrating the skin, so a biodegradable pioneer projectile, which has sufficient hardness to penetrate the skin, is used as an ‘introducer’ for allowing the drug load to enter the skin. The introduction of a foreign material into the skin for the purpose of drug delivery may not be desirable. The foreign material, although biodegradable, may be considered as a new excipient and makes the drug a new drug, which is then subjected to new drug registration process.

It can be seen that the current drug administration techniques including enteral, parenteral and microneedle administrations fail to provide a painless and high-dose drug administration platform. To achieve these three requirements, a device has to be designed to address these three issues, which are inter-dependent.

To start with, a substantial drug loading capacity has to be viable to fulfil most dosage (a few micro grams to a few milligrams), which should be achieved without introducing any new excipients because any new mixture will be treated as a new drug. A typical drug load of these dosages normally amounts to a few nanolitres to a few microliters of volume, which must be transported into the body painlessly. Injecting drug loads at this size into the body will certainly cause pain because the body will need to make space for the drug load which is foreign to the body.

Secondly, the delivery of the drug load should be done as quickly as possible. Currently the parenteral injections are done within 3-5 seconds and the new delivery system should not take more than that because the psychological stress under which a patient is subjected may be too huge and one second more may be unacceptable to the patient. Lastly and most importantly, the rapid delivery of substantial drug load has to be carried out painlessly, otherwise such a device is no better than current needles and syringes.

There is a long-felt need in administering drug to a body painlessly, rapidly, and completely. The present invention seeks to provide a solution for drug administration to the body which is painless, rapid and high dosing.

### SUMMARY OF THE INVENTION

The present invention provides a solution for addressing the design requirements for delivering drug rapidly and painlessly with high and complete dose, which cannot be achieved by microneedles or conventional needles. The present invention relates to a drug implant device which delivers a drug load to a body painlessly. The present invention achieves the drug implantation by adopting two principles: (1) rapid perpendicular insertion of fine cannula is painless and that (2) pain is incurred only when the occupied volume caused by the implant process is increased. Therefore, instead of inserting a cannula and injecting a volume of drug, which increases the additional volume for accommodating the additional volume of the drug during the injection process (i.e. the occupied volume), the present invention retracts the cannula in order to dispose expose the drug into the body. The retraction of cannula does not increase the occupied volume therefore incurs no pain. (In fact, it reduces the occupied volume of the implant process.)

The present invention involves essentially rapid perpendicular insertion of a small amount of drug into the body intradermally, subcutaneously or intramuscularly in a painless manner. In the first preferred embodiment 100, the present invention comprises a fine cannula 300, which has a beveled tip for rapidly penetrating the body, a drug load 320 and an inner rod 340, wherein the drug load 320 and the inner rod 340 are slidably disposed within the cannula 300 and that the drug load 320 is disposed at the beveled end of the cannula 300 (i.e. the forward position) while the inner rod 340 is disposed adjacent to the drug load 320 (i.e. the rearward position). When the first preferred embodiment 100 is in use, the cannula 300, the drug load 320 and the inner rod 340 are rapidly inserted together into the body (in a substantially perpendicular manner in order to eliminate pain) so that the drug load 320 is transported to the desired depth, after which the cannula 300 is retracted while the inner rod 340 and the drug load 320 remain stationary relative to the cannula 300, thereby disposing the drug load 320 at the desired depth of the body, and after which the cannula 300 and the inner rod 340 are fully removed from the body.

The first preferred embodiment 100 may be used with a spring applicator 500 for achieving the desired insertion speed. The spring applicator 500 comprises a slidable casing 520, a transparent cap 540, a returning spring 560, an actuation spring 580, a button 600, and a vault 620. When in operation, device 100 is inserted into spring applicator 500 and is attached to the vault 620. Next, the slidable casing 520 is pulled backward to compress the actuation spring 580, the returning spring 560 will return the slidable casing 520 back to its original position. Lastly, button 600 is depressed to release the vault 620 and the actuation spring 580 will propel the device 100 with the desired speed for rapid insertion.

In the second embodiment, the drug implanting device 1000, a simplified version of the first preferred embodiment is provided. The drug implanting device 1000 comprises a top casing 1100a, a bottom casing 1100b, a lever button 1200 disposed on the top casing 1100a, a compression spring 1300, a cannula 1400, a cannula holder 1500 on which the cannula 1400 is fastened, an inner rod 1600 slidably disposed within the cannula 1600, a rod stopper 1700 on which the inner rod 1600 is fastened, and a drug load 1800 disposed within the tip of the cannula 1400.

### BRIEF DESCRIPTIONS OF THE DRAWINGS

FIG. 1 shows the three-dimensional view of the preferred embodiment of the present invention

FIG. 2 shows the front view of the preferred embodiment of the present invention with the cap removed.

FIG. 3 shows the steps of implanting a drug into a body by the first preferred embodiment of the present invention.

FIG. 4 shows the close-up section view of the first preferred embodiment of the present invention after the cannula penetrates into the body.

FIG. 5 shows the close-up section view of the first preferred embodiment of the present invention after the cannula retracts from the body, exposing the drug in the body.

FIG. 6 shows the close-up section view of the first preferred embodiment of the present invention after the cannula and the inner rod retracts from the body, implanting the drug in the body.

FIG. 7 shows a perspective view of the first preferred embodiment when used with a spring-loaded applicator.

FIG. 8 shows a section view of the first preferred embodiment of the present invention when used with a spring-loaded applicator.

FIG. 9 shows a perspective view of the second preferred embodiment of the present invention.

FIG. 10 shows an exploded view of the second preferred embodiment of the present invention.

FIG. 11 shows a section view of the second preferred embodiment of the present invention.

FIG. 12 (a) to (d) shows the operation of the second preferred embodiment of the present invention.

### DETAILED DESCRIPTION OF THE INVENTION

The present invention aims to provide a painless means to deliver a sizeable drug load into the body. As discussed previously, the injection method is painful, partially due to the insertion of the needle into a body, but more significantly due to injecting a finite volume of drug into the body, which has to make space for that finite volume. The present invention achieves its objectives by two principles. The first principle is that when a cannula is rapidly inserted into the body in a substantially perpendicular manner, provided that the size is small enough, i.e. gauge size of 27 G to 34 G, i.e. with outer diameter between 0.4 mm-0.18 mm and that the insertion speed exceeds 1 m/s, the insertion of the cannula into the body is quite painless. Therefore, the present invention incorporates a rapid perpendicular insertion of cannula to eliminate pain due to needle insertion.

The second principle is that pain is incurred only when the occupied volume caused by the implantation is increased; for example, injecting liquid drug into the body increases the occupied volume as the body needs to make space for the liquid, which causes pain. Instead of injecting any drug, which causes the body to make up the occupied volume for the drug (which is very painful), the present invention pre-loads a drug load 320 in the cannula 300 and rapidly inserts the drug-loaded cannula 300 into a body, after which the cannula 300 is retracted while keeping the drug load 320 in the body. This is achieved by having an inner rod 340 which is disposed in the cannula 300 to hold the drug load 320 in place during the retraction of the cannula 300. In this way, the occupied volume caused by the insertion of the cannula 300 does not increase, but it actually decreases after the cannula 300 is retracted, causing no pain. Once the drug load 300 is disposed in the body, it is wetted by the interstitial fluid in the tissue and is fused to the tissue in the body and will not leave the body easily. Finally, the cannula 300 and the inner rod 340 are removed from the body completely.

It is worth noting that while the insertion of the cannula 300 requires high speed, such as 1 m/s or more, the retraction of cannula 300 requires much lesser speed to prevent the drug load 320 from ‘splashing’ when the cannula 300 retracts. The reason is that the drug load 320 is always in physical contact with the inner surface of the cannula 300, if the cannula 300 retracts in high speed, the surface friction will pull the drug load 320 together, the consequences are a portion of drug may stay in the cannula, and that the drug load 320 may be dispersed to other undesirable depths, causing the drug delivery un-controllable. The retraction speed can be achieved by reducing the actuation speed provided by a compression spring 280. The speed reduction method may be employing a sliding piston that remains in good contact with the stationary surface during sliding, dampening the initial actuation force of the compression spring 280. The sliding pistons are made of silicone rubber, or any material that is able to provide firm contacts between sliding surfaces. The retraction speed for the cannula should be less than 20 mm/s to ensure good implant quality.

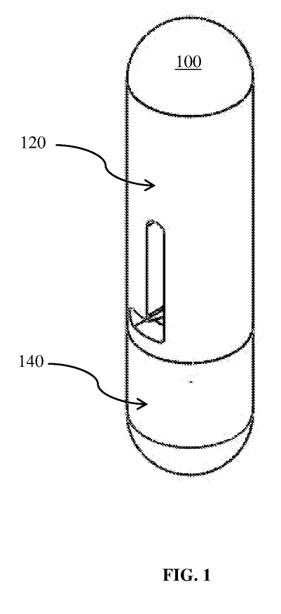

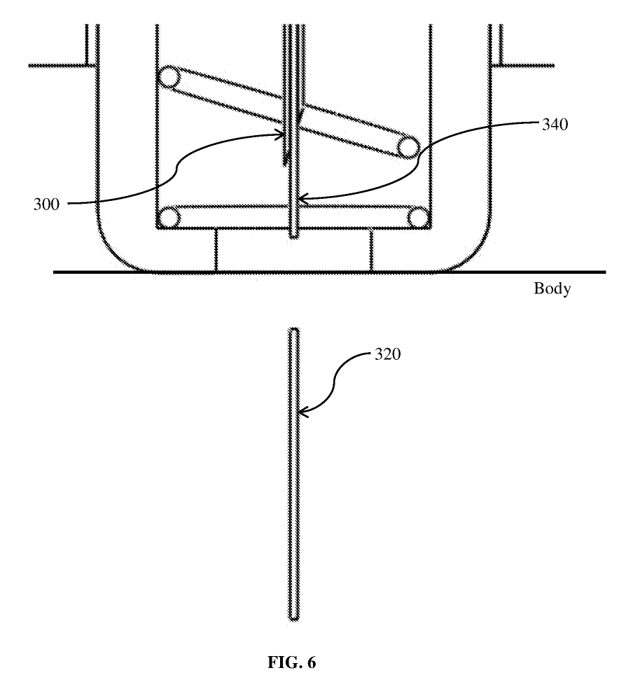

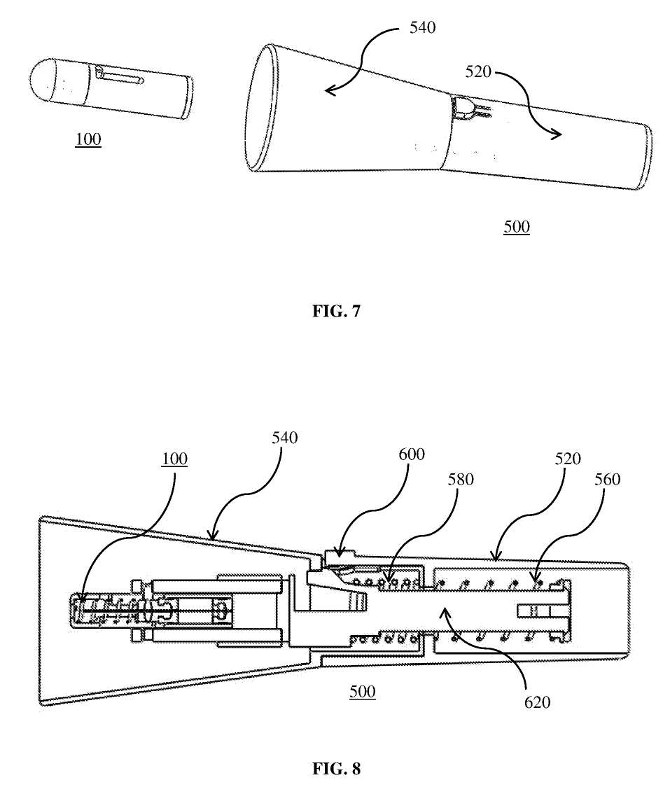

The present invention incorporating these two principles can be represented by two preferred embodiments, which are discussed in the following paragraphs. FIGS. 1-6 describe the first preferred embodiment and FIGS. 7 and 8 describe the second preferred embodiment.

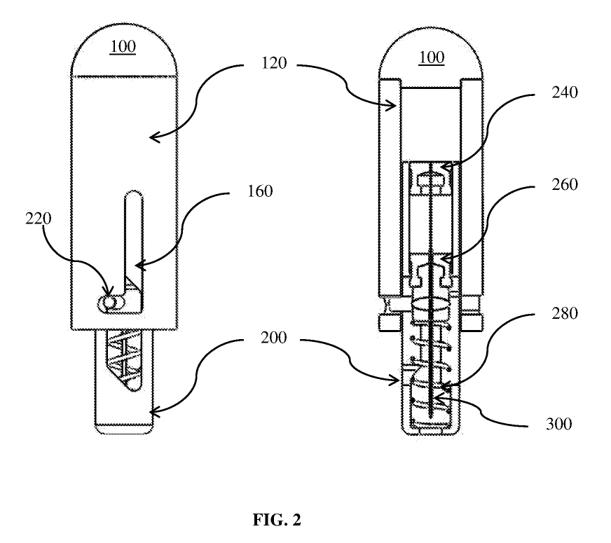

Now, we will describe the first preferred embodiment of the present invention. In FIG. 1, a perspective view of the device (first preferred embodiment) 100 is presented. The first preferred embodiment 100 comprises a housing 120 and a protective cap 140, which is removed when the device is in use. FIG. 2 shows a front view of the device 100 without the protective cap 140. In this figure, the device 100 further comprises a trigger 200, a slider 220, a first piston 240, a second piston 260, a compression spring 280, a cannula 300, which has a beveled tip pointing forward, i.e. pointing away from the housing 120, and a sliding slot 160 disposed on the housing 120. Although not shown in the figure, it is important to know that there is an inner rod 340 and a drug load 320 disposed within the cannula 300. The first piston 240 is fastened to the inner rod 340 and the first piston 240 will resist any unwanted movement due to its firm contact with the housing 120. Similarly, the second piston 260 is fastened to the cannula and the second piston 260 will resist any unwanted movement due to its firm contact with the housing 120. The material for making the pistons is silicone rubber, or any material that remains in good contact between sliding surfaces.

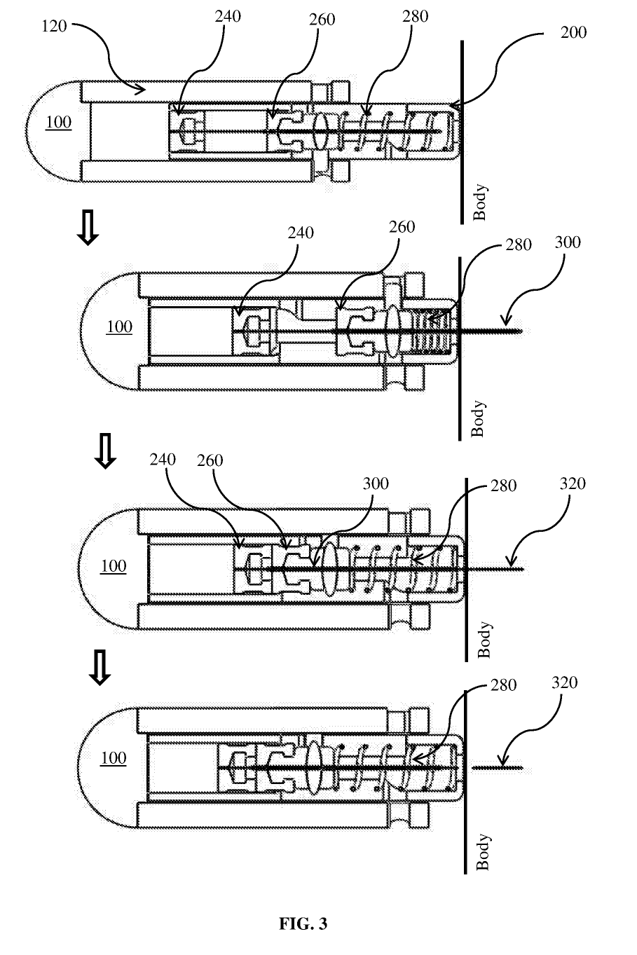

The device 100 relies on manual insertion. FIG. 3 shows how the device 100 operates for implanting a drug load into the body painlessly. First, the protective cap 140 (not shown) is removed to expose the trigger 200, and the device 100 is brought near to a skin site with the trigger 200 pointing at the skin site. Next, the device 100 is compressed against the skin; this action pushes the trigger 200 rearward and at the same time rapidly inserts the cannula 300 in to the body. This rapid insertion by manual compression can be achieved by providing a thrust to the skin site while holding the device 100. The slider 220, to which the cannula 300 is fastened, which is latched originally, resists the penetration force exerted on the cannula 300. At the same time, the first piston 240, which is fastened to the inner rod 320 (not shown) and the second piston 260, which is fastened to the cannula 300, are held firmly in their respective position by surface friction. The pistons are made of silicone rubber or any other material which provides excellent compressibility and surface friction as seen in typical syringe plunger.

The trigger 200 serves to conceal the cannula 300 when not in use. As the trigger 200 continues to be pushed rearward, it engages and unlatches the slider 220; as a result, the compressed compression spring 280 releases its potential energy and pushes rearward the slider 220, which is fastened to the second piston 260 and the cannula 300, sliding on the sliding slot 160. This action retracts the cannula 300 while it is still in the body, disposing the drug load 320 to the body. The compression spring 280 continues to push the slider 220 and the second piston 260 rearward until the second piston 260 hits the first piston 240, after which both pistons 240, 260 move rearward together. As the two pistons 240, 260 move rearward together, the cannula 300 and the inner rod 340 retract from the body together completely. This completes the drug implant process. The implanted drug load is properly disposed in the desired depth, normally within penetration depth of 1 mm-25 mm under the skin.

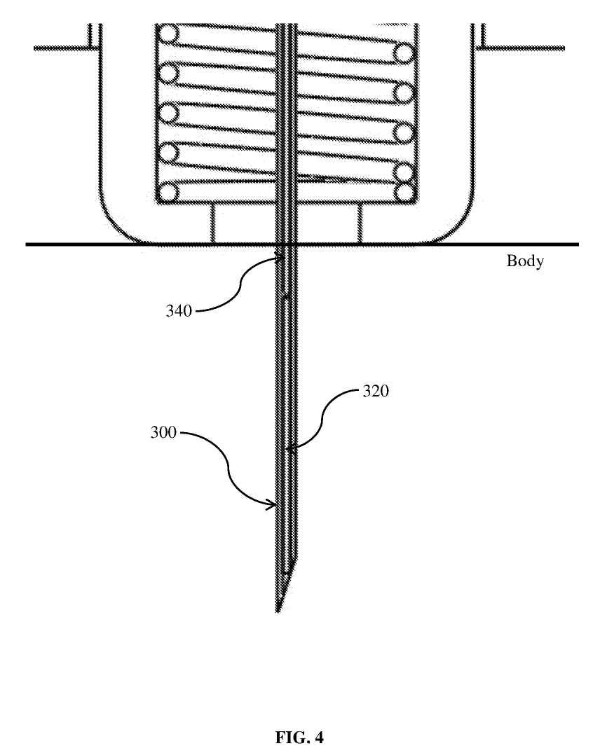

It is vital to understand the exact mechanism of how the implant of drug load into a body can be achieved. FIGS. 4 to 6 show the close-up schematic diagrams for implanting a drug load into a body using the device 100. FIG. 4 shows the device after the cannula is inserted into the body. As shown in FIG. 4, the cannula 300 is inserted in the body with depth of 1 mm-25 mm (intradermal to intramuscular range), the drug load 320 is disposed within the cannula 300 near the tip (i.e. the forward position) and the inner rod 340 is disposed within the cannula 300 and right behind the drug load 320 (i.e. the rearward position). The cannula 300 can be made of typical hypodermic needle with a beveled tip and the needle size (gauge size) ranges from 27 G-34 G, i.e. with outer diameter between 0.4 mm-0.18 mm and inner diameter between 0.2 mm-0.1 mm. The cannula 300 is fastened to the second piston 260 and the slider 220. Two main functions of the cannula 300 are to penetrate the body and to carry the drug load 320 to the desired depth.

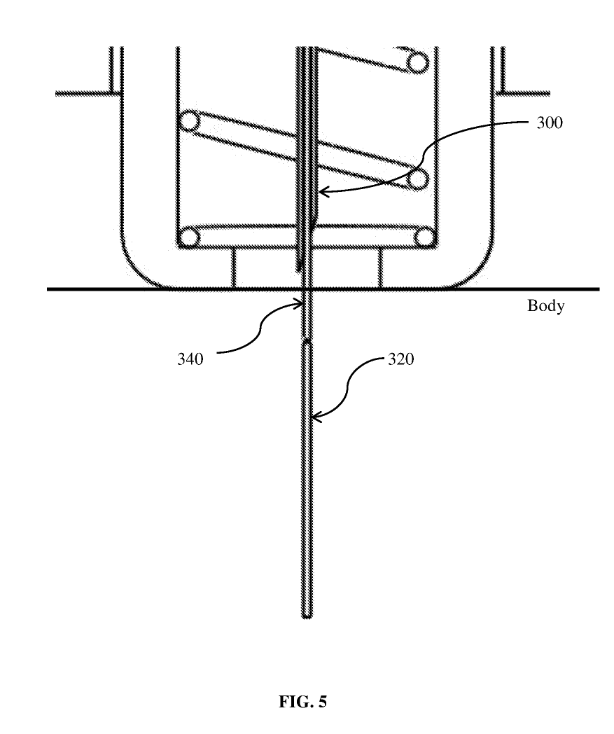

FIG. 5 shows the state after the cannula 300 is retracted from the body, disposing the drug load 320. It can be seen that the inner rod 340 which remains stationary prevents the drug load 320 from sticking to the cannula 300. The inner rod 340 is a metal wire or plastic rod with a diameter the same as the inner diameter of the cannula 300, which is fastened to the first piston 240. FIG. 6 shows the state after both the cannula 300 and the inner rod 340 are retracted from the body, leaving the drug load 320 in the body. The drug load 320 is made of solid drug with total volume of 0.16 uL-0.63 uL, or 0.16 mg-0.63 mg of active pharmaceutical ingredient (this is a very rough conservative estimate).

The first preferred embodiment of the present invention can be used with a spring loaded applicator, which is shown in FIGS. 7 and 8. In this configuration, the first preferred embodiment uses a spring applicator 500 to propel the device 100 toward the body for consistent and repeatable insertion speed. The spring applicator 500 comprises a slidable casing 520, a transparent cap 540, a returning spring 560, an actuation spring 580, a button 600, and a vault 620. When in operation, device 100 is loaded into spring applicator 500 and is attached to the vault 620. Next, the slidable casing 520 is pulled backward to compress the actuation spring 580, and subsequently the returning spring 560 will return the slidable casing 520 back to its original position. Then, the spring applicator 500 is pointed to a body site with the transparent cap 540 resting on the skin site such that the spring applicator 500 is largely perpendicular to the body site. Lastly, the button 600 is depressed to release the vault 620 and the actuation spring 580 propels the device 100 towards the body with the desired speed (i.e. at least 1 m/s) for achieving consistent and repeatable rapid insertion. Once the device 100 is propelled to hit the body and the cannula is inserted into the body, the retraction of the cannula 300 and the inner rod 340 will automatically be carried out as shown in FIG. 3 and FIGS. 4-6.

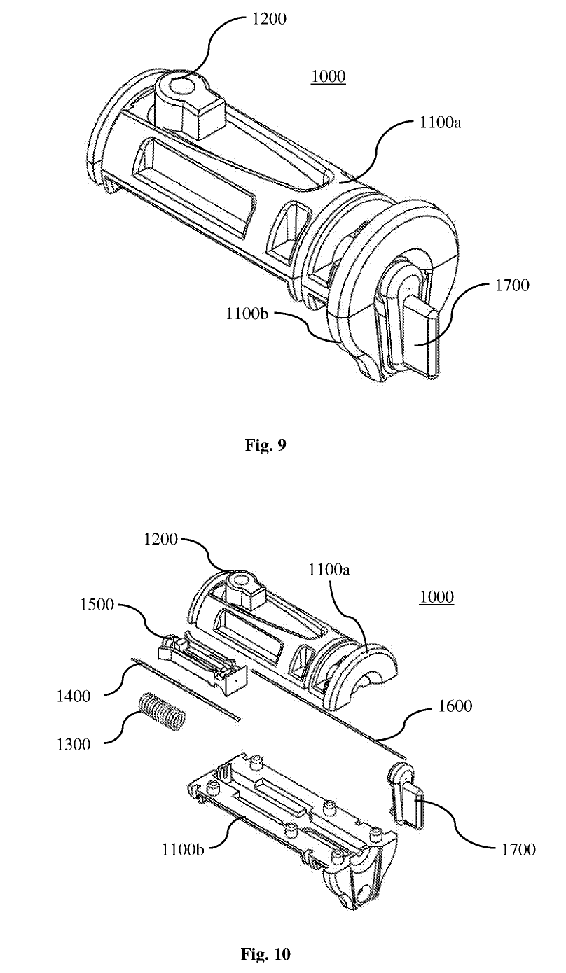

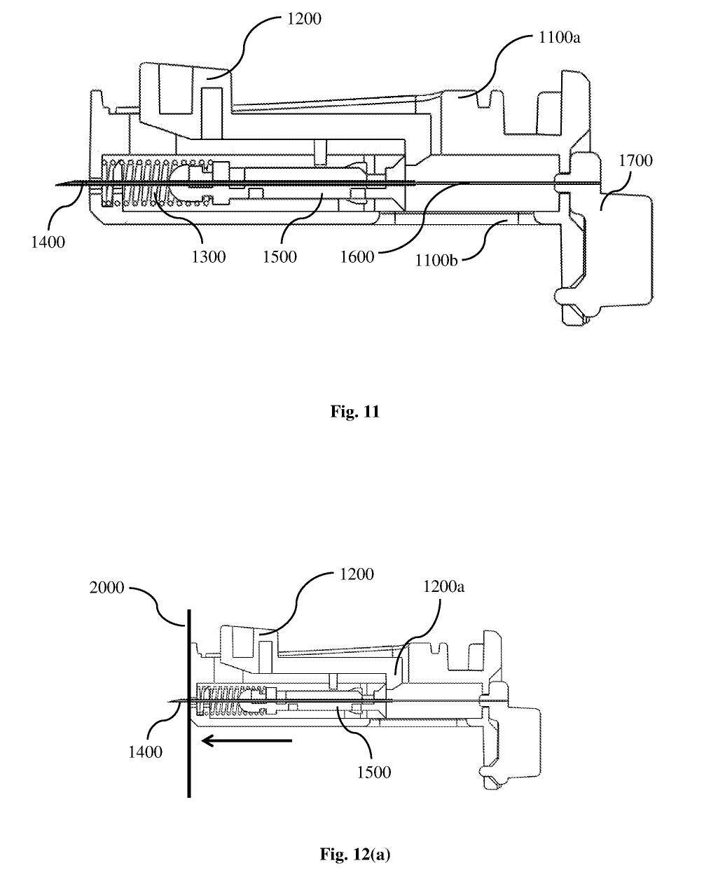

There is a need to provide a simpler device to carry out the drug implant function in the present invention. FIG. 9 shows the perspective view of the second preferred embodiment of the present invention, the drug implanting device 1000, which is a simplified version of the first preferred embodiment. FIG. 10 shows the exploded view of the drug implanting device 1000. The drug implanting device 1000 comprises a top casing 1100a and a bottom casing 1100b, a lever button 1200 disposed on the top casing 1100a, a compression spring 1300, a cannula with a beveled tip 1400, a cannula holder 1500 on which the cannula with a beveled tip 1400 is fastened, an inner rod 1600 slidably disposed within the cannula with a beveled tip 1600, a rod stopper 1700 on which the inner rod 1600 is fastened, and a drug load 1800 disposed within the tip of the cannula with a beveled tip 1400. The drug load 1800 is disposed at the tip of the cannula 1400 and the inner rod 1600 is disposed adjacent to the drug load 1800 within the cannula 1400 such that drug load 1800 is held stationary by the inner rod 1600 when the cannula with a beveled tip 1400 is retracted, thereby disposing the drug load 1800 in the skin 2000.

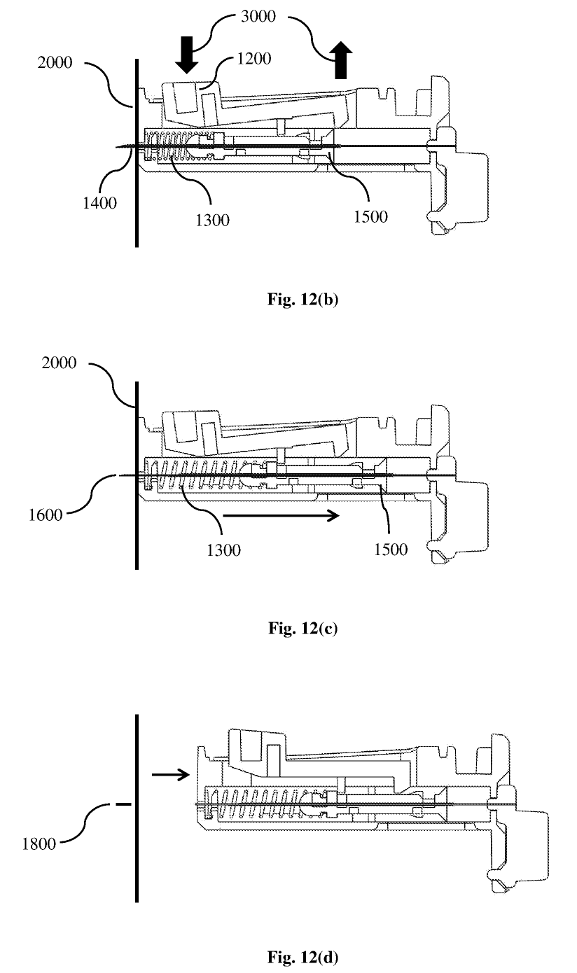

FIG. 12 (a) to (d) shows the operation of the drug implanting device 1000. In the initial stage, the cannula 1600 is exposed outside the drug implanting device 1000. As shown in FIG. 12(a), the cannula 1600 is rapidly inserted into the skin 2000 by hand so that the drug load 1800 is buried in the skin 2000. Next, as shown in FIG. 12(b), the lever button 1200 is depressed, causing a turning moment 3000, thereby unlatching the lever latch 1200a from the cannula holder 1500, which in turn releases the compressed spring 1300. The compressed spring 1300 pushes the cannula 1400 and the cannula holder 1500 backward so as to expose the drug load 1800 in the skin 2000. During this retraction of cannula 1400, the inner rod 1600 is held stationary by the rod stopper 1700. Finally, the inner rod 1600 and the cannula 1400 are removed from the skin site, leaving behind the drug load 1800 in the skin.

1. A device for implanting a drug in the body intradermally, the device comprising a cannula with a beveled tip, an inner rod and a drug load,
a. wherein the drug load and the inner rod are slidably disposed within the cannula and that the drug load is disposed at the beveled end of the cannula (forward position) while the inner rod is disposed adjacent to the drug load (rearward position);
b. wherein the drug load is implanted into the body intradermally by first inserting the cannula comprising the inner rod and the drug load into the body in a substantially perpendicular manner, then retracting the cannula by sliding it on the inner rod thereby disposing the drug load into a body, and finally retracting both the cannula and the inner rod completely from the body.

2. A drug implant device in claim 1, wherein the speed for inserting the cannula is at least 1 m/s.

3. A drug implant device in claim 1, wherein the speed of retracting the cannula is less than or equal to 20 mm/s.

4. A drug implant device in claim 1, wherein the outer diameter of the cannula is 0.4 mm-0.18 mm.

5. A drug implant device in claim 1, wherein the drug load is implanted within 25 mm under the skin.

6. A drug implant device in claim 1, wherein the speed for inserting the cannula is provided by a spring-loaded applicator.

7. A method of implanting a drug into the body intradermally and painlessly, the method comprising:
a. Firstly, preparing a drug implant device comprising a cannula with a beveled tip, a drug load and an inner rod, wherein the drug load and the inner rod are slidably disposed within the cannula, and that the drug load is disposed at the beveled end of the cannula (forward position) while the inner rod is disposed adjacent to the drug load (rearward position);
b. Secondly, inserting the cannula comprising the inner rod and the drug load into the body intradermally in a substantially perpendicular manner with at least a predetermined insertion speed,
c. Thirdly, retracting the cannula by sliding it on the inner rod thereby disposing the drug load, and
d. Finally, retracting both the cannula and the inner rod completely from the body at a predetermined speed.

8. A method of implanting a drug into the body painlessly in claim 7, wherein the predetermined speed for inserting the cannula is at least 1 m/s.

9. A method of implanting a drug into the body painlessly in claim 7, wherein the speed of retracting the cannula is less than or equal to 20 mm/s.

10. A method of implanting a drug into the body painlessly in claim 7, wherein the outer diameter of the cannula is 0.4 mm-0.18 mm.

11. A method of implanting a drug into the body painlessly in claim 7, wherein the drug load is implanted within 25 mm under the skin.

12. A method of implanting a drug into the body painlessly in claim 8, wherein the speed for inserting the cannula is provided by a spring-loaded applicator.

13. A drug implant device in claim 1, the depth of insertion of the cannula is 1 mm-25 mm below the skin of a body

14. A method of implanting a drug into a body intradermally and painlessly in claim 8, wherein the depth of the insertion of the cannula is 1 mm-25 mm below the skin.
